(19) United States
(12) Patent Application Publication
(10) Pub. No.: US 2026/0369420 A1
(43) Pub. Date: Nov. 19, 2026

(54) PREDICTION DEVICE, NON-TRANSITORY COMPUTER-READABLE MEDIUM, METHOD OF GENERATING PREDICTION MODEL, AND DEVICE FOR GENERATING PREDICTION MODEL

(71) Applicant: NIHON KOHDEN CORPORATION, Tokyo (JP)

(72) Inventors: Daisuke HORIGUCHI, Saitama (JP); Wataru MATSUZAWA, Saitama (JP); Hiroto SANO, Saitama (JP); Yuichiro SAWANO, Saitama (JP)

(73) Assignee: NIHON KOHDEN CORPORATION, Tokyo (JP)

(21) Appl. No.: 19/673,314

(22) Filed: May 11, 2026

(30) Foreign Application Priority Data
May 19, 2025 (JP) 2025-083158

(51) Int. Cl. G16H 50/30; G06N 20/00

(52) U.S. Cl. CPC G16H 50/30; G06N 20/00

(57) ABSTRACT

An interface receives explanatory variable data corresponding to a time series of medical information of a subject. A processor acquires response variable data corresponding to a time length until a medical event occurs in the subject or a likelihood that the medical event occurs within a specific time period, each of which is predicted by a prediction modal based on a regression algorithm in response to reception of the explanatory variable data. The processor causes an output device to output information corresponding to the response variable data.

### CROSS-REFERENCE TO RELATED APPLICATION

The present application is based on Japanese Patent Application No. 2025-083158 filed on May 19, 2025, the entire contents of which are incorporated herein by reference.

### BACKGROUND

The presently disclosed subject matter relates to a prediction device configured to predict a likelihood of a clinical event occurring in a subject. The presently disclosed subject matter also relates to a non-transitory computer-readable medium having stored a computer program adapted to be executed by a processor that is to be installed in the prediction device. The presently disclosed subject matter also relates to a method and device for generating a prediction model adapted to be used by the processor.

Japanese Patent Publication No. 2014-054391A discloses a Holter electrocardiograph configured to acquire an electrocardiogram that is an exemplary physiological parameter of a subject. The data corresponding to a measured waveform of the electrocardiogram is received by an information processing device that is disposed in a remote place. The information processing device is configured to analyze the data to detect an abnormality in the measured waveform, and to identify onset of heart failure or the like, that is an exemplary clinical event occurring in a subject.

### SUMMARY

There is a need to more accurately predict a likelihood of a clinical event occurring in a subject.

An illustrative aspect of the presently disclosed subject matter provides a prediction device, comprising:
an interface configured to receive explanatory variable data corresponding to a time series of medical information of a subject; and
a processor configured to:
   acquire response variable data corresponding to a time length until a medical event occurs in the subject or a likelihood that the medical event occurs within a specific time period, each of which is predicted by a prediction modal based on a regression algorithm in response to reception of the explanatory variable data; and
   cause an output device to output information corresponding to the response variable data.

An illustrative aspect of the presently disclosed subject matter provides a non-transitory computer-readable medium having stored a computer program adapted to be executed by a processor installed in a prediction device, the computer program being configured to cause, when executed, the prediction device to:
receive explanatory variable data corresponding to a time series of medical information of a subject;
acquire response variable data corresponding to a time length until a medical event occurs in the subject or a likelihood that the medical event occurs within a specific time period, each of which is predicted by a prediction modal based on a regression algorithm in response to reception of the explanatory variable data; and
cause an output device to output information corresponding to the response variable data.

An illustrative aspect of the presently disclosed subject matter provides a method of generating a prediction model, comprising:
preparing training data corresponding to a time series of medical information associated with a time point when a medical event occurs; and
performing machine learning with the training data to generate a prediction model that is configured to apply a regression algorithm with respect to the time series of the medical information to predict a time length until a medical event occurs in a subject or a likelihood that the medical event occurs within a specific time period.

An illustrative aspect of the presently disclosed subject matter provides a device for generating a prediction model, comprising:
an interface configured to receive training data corresponding to a time series of medical information associated with a time point when a medical event occurs; and
a processor configured to perform machine learning with the training data to generate a prediction model that is configured to apply a regression algorithm with respect to the time series of the medical information to predict a time length until a medical event occurs in a subject or a likelihood that the medical event occurs within a specific time period.

As a prediction model for predicting whether a medical event occurs, a model based on a classification algorithm is known. In this case, since a binary response variable indicating whether the medical event occurs is given, it is necessary to define whether each data constituting the time series of the explanatory variable is positive (related to the medical event) or negative (not related to the medical event). To perform this definition, a time window may be employed that has a specific time duration prior to the occurrence of the medical event. In other words, explanatory variable data that fall within the time window is regarded as positive.

However, since it is general that a value of the explanatory variable such as the measured value of the physiological parameter gradually changes toward the time point when the medical event occurs, the behavior of the measured value would not exhibit a sudden change even though it falls within the time window. Accordingly, particularly in the vicinity of a boundary of the time window, a positive label may be assigned even if the data is unrelated to the medical event, or a negative label may be assigned even if the data is related to the medical event.

In addition, a numerical value outputted from the prediction model based on the classification algorithm represents a probability that the explanatory variable data is related to the medical event (falls within the time window). In this case, the output result may be variously interpreted as follows: Should an intervention be considered even when the probability is as low as 1%?; Should an intervention be necessary if the probability is as high as 50%?; Should an intervention not be undertaken unless the probability reaches 100%?; and so on. Even when the occurrence probability of the medical event is 100%, it is impossible to distinguish whether the medical event occurs after one minute or after the elapse of a time period corresponding to the time window.

According to the configuration of each of the illustrative aspects, the response variable data that is outputted from the prediction model based on the regression algorithm tends to exhibit continuous and progressive change toward the time point when the medical event occurs. In this case, even a slight change of the explanatory variable can be reflected in the difference of the response variable. As a result, it is possible to more accurately predict a likelihood of a clinical event occurring in a subject.

### DESCRIPTION OF THE DRAWINGS

FIG. 1 illustrates a functional configuration of a prediction device according to one exemplary embodiment.

FIG. 2 is a diagram for explaining advantages of a prediction model based on regression algorithm.

FIG. 3 illustrates a functional configuration of a model generating device according to one exemplary embodiment.

FIG. 4 illustrates configuration of training data adapted to be used to generate a prediction model of FIG. 3.

FIG. 5 illustrates configuration of training data according to a first comparative example.

FIG. 6 illustrates configuration of training data according to a second comparative example.

### DESCRIPTION OF EMBODIMENTS

Exemplary embodiments are described in detail below with reference to the accompanying drawings.

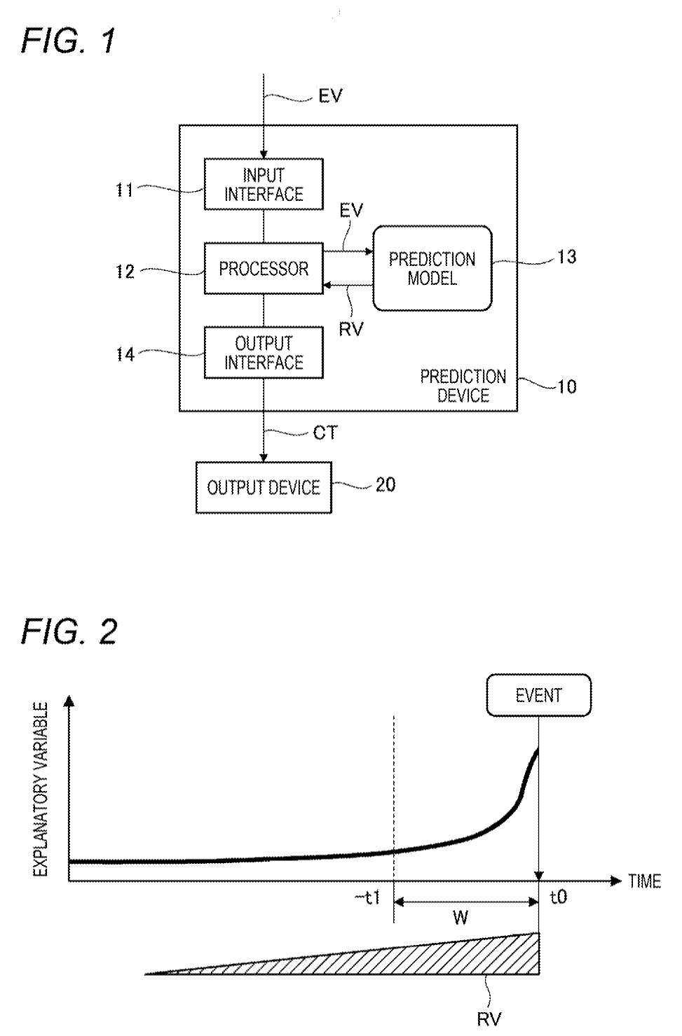

FIG. 1 illustrates a functional configuration of a prediction device 10 according to an exemplary embodiment. The prediction device 10 may be installed in a place that is remote from a device that acquires a value of a physiological parameter of a subject, or may be incorporated in such a device.

The prediction device 10 includes an input interface 11. The input interface 11 is a hardware interface that receives explanatory variable data EV corresponding to a time series of medical information of a subject. The explanatory variable data EV may be provided in the form of analog data or digital data. In the case where the explanatory variable data EV is provided in the form of analog data, the input interface 11 is provided with an adequate conversion circuit including an A/D converter.

In this example, the time series of the medical information of the subject is an exemplary time series of measured values of at least one kind of physiological parameter of the subject. The measured waveform of the physiological parameter may be regarded as a time series of the measured values.

A feature of the time series of the measured values may also be an exemplary time series of medical information. Examples of the feature include an average value, a median value, a mode value, a maximum value, a minimum value, a standard deviation, a frequency component, and the like.

A time series of a score that is calculated based on the measured values of multiple physiological parameters may also be an exemplary time series of medical information. Examples of the score include various early warning scores, indices determined by various academic organizations, and the like.

The prediction device 10 includes a processor 12 and a prediction model 13. The processor 12 is configured to input the explanatory variable data EV accepted by the input interface 11 to the prediction model 13 in order to perform processing for acquiring response variable data RV. The response variable data RV corresponds to a time length until a particular medical event occurs in the subject, or a likelihood that the medical event occurs within a specific time period.

Examples of medical events that may occur in the subject include cardiac arrest, ventricular arrhythmias, prolonged (e.g., 1 minute or more) hypotension, unexpected mortality, transfer to a care unit that can accommodate a higher severity, bleeding with transfusion, tracheal intubation, and abnormal behaviors associated with postoperative delirium.

In other words, the prediction model 13 is a computer algorithm that is configured to, in response to input of a time series of medical information of a subject, predict a time length until a particular medical event occurs in the subject, or a likelihood that the medical event occurs within a specific time period. Specifically, the prediction model 13 is configured to output, , based on a regression algorithm, a continuous value associated with time as the response variable.

The prediction device 10 includes an output interface 14. The processor 12 outputs, from the output interface 14, a control signal CT that causes an output device 20 to output information corresponding to the response variable data RV. The information output from the output device 20 is performed with at least one of a visual manner and an audible manner. Examples of the information output with the visual manner include displaying with a display, projection with a projector, and printing with a printer.

As used herein, the expression "information corresponding to response variable data" may include not only a value itself of the response variable, but also presence or absence of a medical event that can be derived from the value. For example, in a case where a time length until a particular medical event occurs is larger than a threshold, or in a case where a likelihood that the medical event occurs within a specific time period is less than a threshold, information indicating that “the medical event is absent” may be provided.

In other words, the output interface 14 is configured as a hardware interface adapted to output the control signal CT. The control signal CT may be an analog signal or a digital signal in accordance with the specification of the output device 20. In a case where the control signal CT is an analog signal, the output interface 14 provided with an adequate conversion circuit including a D/A converter.

The prediction device 10 and the output device 20 may be independent devices that are disposed apart from each other, or may be incorporated in the same device.

With reference to FIG. 2, advantages that can be achieved by the prediction being performed based on the regression algorithm will be described. FIG. 2 illustrates a time series of measured values of a physiological parameter as an exemplary explanatory variable. It is understood that a medical event occurs at a time point t0, and the measured value significantly changes toward the time point t0.

As a prediction model for predicting whether a medical event occurs, a model based on a classification algorithm is known. In this case, since a binary response variable indicating whether the medical event occurs is given, it is necessary to define whether each data constituting the time series of the explanatory variable is positive (related to the medical event) or negative (not related to the medical event). To perform this definition, a time window W may be employed that has a specific time duration (e.g., 24 hours) prior to the occurrence of the medical event. In other words, explanatory variable data that fall within the time window W is regarded as positive.

However, as illustrated in FIG. 2, since it is general that a value of the explanatory variable such as the measured value of the physiological parameter gradually changes toward the time point t0 when the medical event occurs, the behavior of the measured value would not exhibit a sudden change even though it falls within the time window W. Accordingly, particularly in the vicinity of a boundary of the time window W (a time point -t1), a positive label may be assigned even if the data is unrelated to the medical event, or a negative label may be assigned even if the data is related to the medical event.

In addition, a numerical value outputted from the prediction model based on the classification algorithm represents a probability that the explanatory variable data is related to the medical event (falls within the time window W). In this case, the output result may be variously interpreted as follows: Should an intervention be considered even when the probability is as low as 1%?; Should an intervention be necessary if the probability is as high as 50%?; Should an intervention not be undertaken unless the probability reaches 100%?; and so on.

Even when the occurrence probability of the medical event is 100%, it is impossible to distinguish whether the medical event occurs after one minute or after the elapse of a time period corresponding to the time window W. In order to avoid this problem, it is conceivable to use, for machine learning of the prediction model, explanatory variable data at a time point that is obtained by tracing back a specific time length from the time when the medical event occurs. For example, in a case where an attempt is made to predict probability that the medical event occurs after one hour, a positive label is assigned only to the data 1 hour before the occurrence of the medical event. In this case, since only data at a single time point can be obtained for every medical event, data for a large amount of medical events need to be prepared in order to realize sufficient machine learning.

On the other hand, the response variable data RV that is outputted from the prediction model 13 based on the regression algorithm as in the present exemplary embodiment tends to exhibit continuous and progressive change toward the time point t0 when the medical event occurs, as illustrated in FIG. 2. In this case, even a slight change of the explanatory variable can be reflected in the difference of the response variable. As a result, it is possible to more accurately predict a likelihood of a clinical event occurring in a subject.

As described above, a time length until the occurrence of a particular medical event may be acquired as a continuous response variable associated with time. The information related to the time length as obtained provides following advantages. As an example, in a case where it is known that a prognosis happens to be degraded by an adverse reaction if an intervention is performed more than 6 hours prior to occurrence of a particular medical event for which the prognosis is inherently expected with the intervention, it is possible to establish a protocol in which the intervention is considered if the time length outputted from the prediction model 13 is less than 6 hours. As another example, in a case where it is known that 15 minutes are required for preparing a necessary drug after the occurrence of a certain medical event, it is possible to establish a protocol in which observation is performed while preparing the drug if the time length outputted from the prediction model 13 is less than 15 minutes.

Alternatively, a likelihood that a particular medical event occurs within a specific time period may be acquired as the continuous response variable associated with time. The information related to the likelihood as obtained provides following advantages. For example, in a case where it is known that occurrence probability of a certain medical event linearly increases over time, likelihood that the medical event occurs after one hour can be identified as 50% when the prediction model 13 outputs 100% of likelihood that the medical event occurs after two hours. Accordingly, multiple sets of training data can be prepared for a single medical event.

As used herein, the expression "continuous response variable associated with time" means a response variable that has undergone a transformation by a function using, as a variable, a time length t until a medical event occurs. For example, a reciprocal (1/t) of the time length t may be used as the response variable. In this case, the relative ordering of the likelihood as predicted and the response variable as obtained is consistent. Another example of a function for which such a relationship can be observed is exp (-t). This function is highly suitable for a medical event in which a rapid change of the response variable is expected to occur immediately before the occurrence thereof.

As used herein, the expression "continuous response variable associated with time" intends to include a case where a value of the response variable is clipped to a threshold if the value exceeds the threshold. The response variable that has been subjected to such processing is highly suitable for a medical event in which progressive change can be observed from few hours before the occurrence thereof but no change can be observed before that.

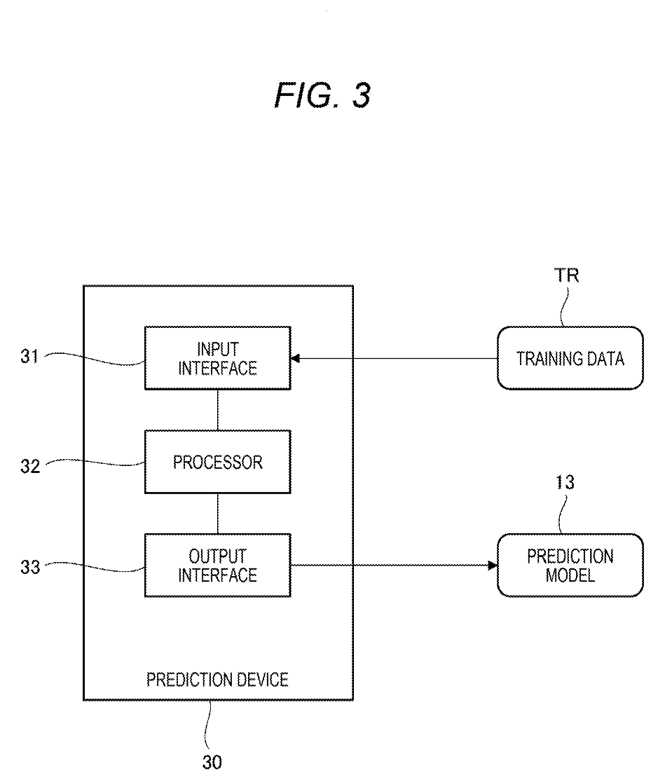

The above-described prediction model 13 is generated by a model generating device 30 illustrated in FIG. 3. In other words, the model generating device 30 is configured to generate a prediction model 13 adapted to be installed in the above-described prediction device 10.

The model generating device 30 includes an input interface 31. The input interface 31 is configured as a hardware interface that receives training data TR. The training data TR includes information corresponding to a time series of medical information associated with a time point when a particular medical event occurs. The training data TR may be provided in the form of analog data or digital data. In the case where the training data TR is provided in the form of analog data, the input interface 31 is provided with an adequate conversion circuit including an A/D converter.

The model generating device 30 includes a processor 32. The processor 32 is configured to generate the prediction model 13 by causing, for example, a neural network to perform machine learning with the training data TR. In this example, a technique related to supervised learning is used to cause the neural network to learn. In the machine learning, an algorithm such as a decision tree, a random forest, a support vector machine, a Bayesian estimation, and a Gaussian hypothesis may be used.

Specifically, the training data TR is configured to include information that teaches what kind of response variable data RV is outputted when what kind of explanatory variable data EV is inputted. The response variable data RV corresponds to a time length until a particular medical event occurs, or a likelihood that the medical event occurs within a specific time period.

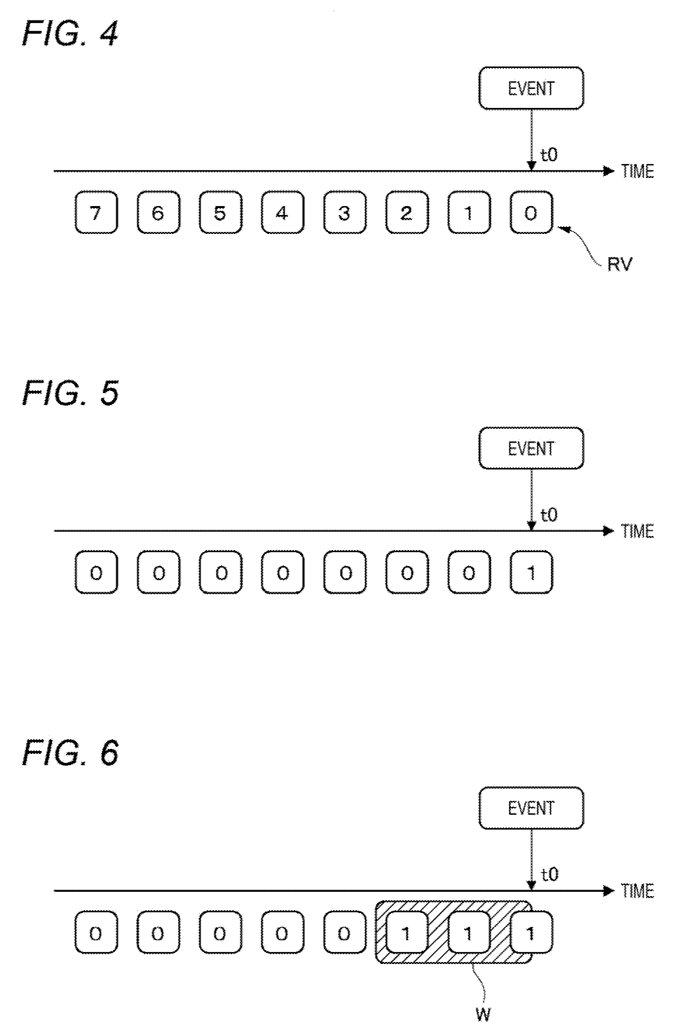

FIG. 4 illustrates a configuration of training data indicating, as the response variable data RV, a time length until a medical event occurs. It is understood that a labelled value corresponding to the response variable RV decreases as a time point t0 approaches. The “t0” represents the time point when the medical event occurs.

FIG. 5 illustrates a configuration of training data according to a first comparative example that may be used for generating a prediction model that is based on a classification algorithm. In this example, only the data at the time point t0 when the medical event occurs are treated as positive.

FIG. 6 illustrates a configuration of training data according to a second comparative example that may be used for generating a prediction model that is based on another classification algorithm. In this example, the data included in the time window W corresponding to a specific time period preceding the time point t0 when the medical event occurs is treated as positive.

As an example, the explanatory variable data EV may correspond to a measured waveform of a physiological parameter that is acquired from a subject. Only a specific section in the measured waveform may be included in the explanatory variable data EV. At least one feature in the measured waveform may be included in the explanatory variable data EV. At least partial combination of measured waveforms of multiple physiological parameters may be included in the explanatory variable data EV. At least one feature in the measured waveforms of the multiple physiological parameters may be included in the explanatory variable data EV.

As illustrated in FIG. 3, the model generating device 30 includes an output interface 33. The output interface 33 is configured as a hardware interface that outputs the prediction model 13 that is generated by the processor 32 in a form that can be installed in the prediction device 10.

According to such a configuration, it is possible to reduce burden related to preliminary design of an explicit rule applied to the prediction that is to be performed by the prediction device 10. In addition, the prediction model 13 can be used in a wide range of implementations of the prediction device 10.

In order to enhance the classification ability of the prediction performed by the prediction device 10, the training data TR may be configured to include explanatory variable data EV in a case where no particular medical event occurs. In this case, such response variable data RV that is distinguished from one to be outputted when the particular medical event occurs may be outputted.

In order to enhance the generalization ability of the prediction performed by the prediction device 10, the training data TR may include non-temporal background information of the subject. Examples of such background information include age, sex, height, weight, past medical history, family history, past examination result, drug history, past intervention events, past medical events, and the like.

In order to utilize the prediction capability of the prediction model 13 that is generated with the training data TR configured as described above, the explanatory variable data EV that is to be received by the prediction device 10 may also include the above-described background information.

It should be noted that the change of the explanatory variable until the occurrence of the medical event may not be monotonous as illustrated in FIG. 2. For example, it is known that a transient hypotension is observed before a medical event of long-term hypotension occurs. In other words, a fact that the blood pressure is currently low may imply that a long-term hypotension likely occurs later regardless of the time length until the medical event occurs.

In view of the above, upon determination of the response variable, correction may be applied to the association between the medical information and the time point when the medical event occurs with a function in which a value x of blood pressure as time-independent medical information and time t are used as variables. Examples of such a function include f(x)/t. The f(x) may be a linear function or a nonlinear function. Examples of f(x) include 1/x, a function whose value changes stepwise as described below in accordance with the value of blood pressure, and the like.

f(x) = 2(x < 65); 1(65 ≤ x < 75); 0.5 (75 ≤ x)

The relationship between f(x) and t for obtaining the function is not limited to basic arithmetic operations.

Each of the processor 12 of the prediction device 10 and the processor 32 of the model generating device 30 having various functions as exemplified above may be implemented by at least one versatile microprocessor configured to cooperate with at least one versatile memory. Examples of the versatile microprocessor include a CPU, an MPU, and a GPU. Examples of the versatile memory include a ROM, and a RAM. In this case, a computer program for implementing the above-described functions may be stored in the ROM. The ROM is an exemplary non-transitory computer-readable medium having stored a computer program. The versatile microprocessor designates at least a part of the program stored in the ROM, loads the designated program in the RAM, and executes the above-described processing in cooperation with the RAM. The computer program may be pre-installed in a versatile memory, or may be downloaded from an external server device with a communication network, and then installed in the versatile memory. In this case, the external server device is an exemplary non-transitory computer-readable medium having stored a computer program.

Each of the processor 12 and the processor 32 may be implemented by at least one exclusive integrated circuitry capable of executing the above-described computer program. Examples of the exclusive integrated circuitry include a microcontroller, an ASIC, and an FPGA. In this case, the above-described computer program is pre-installed in the memory element included in the exclusive integrated circuitry. The memory element is an exemplary non-transitory computer-readable medium having stored a computer program. Each of the processor 12 and the processor 32 may also be implemented by a combination of a versatile microprocessor and an exclusive integrated circuitry.

The foregoing description of various configurations is provided merely by way of illustration to facilitate understanding of the presently disclosed subject matter. Each of the above exemplary configurations may be appropriately modified or combined with another exemplary configuration within the scope of the presently disclosed subject matter.

In the above exemplary embodiment, a time series of at least one of a value and a feature of at least one physiological parameter acquired from a subject is associated with a time point when a particular medical event occurs as a time series of medical information. However, an elapsed time length from hospital admission of a subject, and an elapsed time length from medication or intervention with respect to a subject may also be associated with a time point when a particular medical event occurs as a time series of medical information.

As used herein, the meaning of the term "prediction" may be interpreted so as not to exclude the meaning of each of the terms "inference", "estimation", and "forecasting".

1. A prediction device, comprising:
an interface configured to receive explanatory variable data corresponding to a time series of medical information of a subject; and
a processor configured to:
acquire response variable data corresponding to a time length until a medical event occurs in the subject or a likelihood that the medical event occurs within a specific time period, each of which is predicted by a prediction modal based on a regression algorithm in response to reception of the explanatory variable data; and
cause an output device to output information corresponding to the response variable data.

2. The prediction device according to claim 1,
wherein the medical information is at least one of a value and a feature of at least one kind of physiological parameter acquired from the subject.

3. The prediction device according to claim 1,
wherein the explanatory variable data includes non-temporal background information of the subject.

4. A non-transitory computer-readable medium having stored a computer program adapted to be executed by a processor installed in a prediction device, the computer program being configured to cause, when executed, the prediction device to:
receive explanatory variable data corresponding to a time series of medical information of a subject;
acquire response variable data corresponding to a time length until a medical event occurs in the subject or a likelihood that the medical event occurs within a specific time period, each of which is predicted by a prediction modal based on a regression algorithm in response to reception of the explanatory variable data; and
cause an output device to output information corresponding to the response variable data.

5. A method of generating a prediction model, comprising:
preparing training data corresponding to a time series of medical information associated with a time point when a medical event occurs; and
performing machine learning with the training data to generate a prediction model that is configured to apply a regression algorithm with respect to the time series of the medical information to predict a time length until a medical event occurs in a subject or a likelihood that the medical event occurs within a specific time period.

6. The method according to claim 5,
wherein the training data includes data corresponding to a time series of the medical information in which the medical event does not occur.

7. The method according to claim 5, further comprising:
correcting association between the medical information and the time point with a function in which time-independent medical information and time are used as variables.

8. A device for generating a prediction model, comprising:
an interface configured to receive training data corresponding to a time series of medical information associated with a time point when a medical event occurs; and
a processor configured to perform machine learning with the training data to generate a prediction model that is configured to apply a regression algorithm with respect to the time series of the medical information to predict a time length until a medical event occurs in a subject or a likelihood that the medical event occurs within a specific time period.
